(19) United States
(12) Patent Application Publication
(10) Pub. No.: US 2022/0095977 A1
(43) Pub. Date: Mar. 31, 2022

(54) MONITORING AND PREDICTION SYSTEM OF DIURESIS FOR THE CALCULATION OF KIDNEY FAILURE RISK, AND THE METHOD THEREOF

(71) Applicant: POLITECNICO DI TORINO, Torino (IT)

(72) Inventors: Andrea ANCONA, Torino (IT); Valentina Alice CAUDA, Torino (IT); Erika MANTI, Torino (IT)

(73) Assignee: POLITECNICO DI TORINO, Torino (IT)

(21) Appl. No.: 17/427,034

(22) PCT Filed: Nov. 21, 2019
(86) PCT No.: PCT/IB2019/060017

§ 371 Date: Jul. 29, 2021

(30) Foreign Application Priority Data
Jan. 30, 2019 (IT) 102019000001365

(51) Int. Cl. A61B 5/20; A61B 5/00; A61B 10/00; G16H 10/60

(52) U.S. Cl. CPC A61B 5/201; A61B 5/207; A61B 5/7275; A61B 2562/0252; A61B 5/7264; A61B 10/007; G16H 10/60; A61B 5/0002

(57) ABSTRACT

A monitoring system, and related monitoring and predicting methods of a diuresis for a calculation of a risk of onset of renal failure of a patient, including a device, wherein the device includes a first algorithm for recording, storing, comparing and processing measurements of a urine container and a second algorithm for predicting future measurements of the urine container and a level of a kidney failure risk associated with the future measurements of the urine container. The monitoring system, and relevant monitoring and predicting methods, of a biological fluid for predicting a state of health of the patient, including a device, wherein the device includes a first algorithm for recording, storing, comparing and processing measurements of a biological fluid container and a second algorithm for predicting future measurements of the biological fluid container and the state of health of the patient associated with the future measurements of the biological container.

### CROSS REFERENCE TO THE RELATED APPLICATIONS

This application is the national stage entry of International Application No. PCT/IB2019/060017, filed on Nov. 21, 2019, which is based upon and claims priority to Italian Patent Application No. 102019000001365 filed on Jan. 30, 2019, the entire contents of which are incorporated herein by reference.

### TECHNICAL FIELD

The present invention relates to the medical field and, in particular, to the monitoring and the prediction of vital parameters of a patient for the purpose of early diagnosis of possible alterations of his state of health.

Specifically, the present invention relates to a system and method for the early diagnosis of acute kidney failure in hospitalized and catheterized patients; such early diagnosis is obtained through the continuous monitoring of the patient's diuresis and the automatic and instant evaluation and communication to the attending physician of the critical stage of the patient's kidney function.

In general, the present invention relates to a system and method for early diagnosis obtained through the continuous monitoring of biological fluid of the patient to establish his/her state of health.

The preferred fields of application of the present invention are the hospital and nursing clinic, intensive care, nephrology, urology, cardiology, transplant surgery and the like.

### BACKGROUND

The investigation of the proper functioning of the kidneys and the monitoring of diuresis are often undervalued findings in the management of hospitalized patients. Due to the ageing of the population, increasingly efficient surgical and resuscitation practices, acute kidney injury (AKI) is currently a syndrome that can be found in intensive care of all kinds.

AKI is defined as a kidney injury that can reach failure, and that can have results even remotely over time; the term identifies the entire spectrum of acute kidney injuries, recognizing that the decline in renal function is often secondary to a lesion that causes functional or structural alterations in the kidneys.

Furthermore, intensive care is not the only field in which this syndrome can occur; numerous pieces of evidence of clinical data (see KDIGO, Clinical Practice Guideline for Acute Kidney Injury, Kidney International Supplements (2012) Vol. 2, Suppl. 1, pages 1-138) showed that a large number of patients, although not requiring dialysis, developed AKI during hospitalization. The Societá Italiana di Nefrologia (Italian Society of Nephrology) defines AKI as a sudden reduction in kidney function which includes acute kidney failure, (Insufficienza Renale Acuta—IRA), and multiple pathological conditions affecting kidney structure and function.

Clinical data, including the study by Uchino et al. (see Uchino S., Bellomo R., Goldsmith D., et al., An assessment of the RIFLE criteria for acute renal failure in hospitalized patients, Crit. Care Med. 2006; 34: 1913-1917), have pointed out that even moderate and reversible forms of AKI can carry a risk of increased mortality; actually, it has been demonstrated that patients classified with a less severe RIFLE risk level, RIFLE-R (Risk), shown a mortality 2.5 times higher than patients not classified as prone to kidney failure according to the RIFLE criteria. Also, it has been estimated (see Mandelbaum M. et al. “Outcome of critically ill patients with acute kidney failure using the Acute Kidney Injury Network criteria.” Crit. Care Med. (2011) 39(12):2659-64) that more than 5% of all hospitalized patients and about 50% of patients admitted to intensive care suffer from AKI, and that in Italy more than 400 thousand people suffer from acute kidney injury every year: it has also been estimated (see Macedo E., Malhotra R., Bouchard J., Wynn S. K., Mehta R. L. “Oliguria is an early predictor of higher mortality in critically ill patients” Kidney International (2011) 80, 760-767) that the onset of acute kidney failure in hospitalized patients in intensive care increases mortality by five times and requires an average of additional twenty-seven days of hospitalization, with a consequent increase of the estimated health care expenses up to 4,000 euros/patient.

AKI results, therefore, a common, harmful, but potentially treatable condition, in which an acute reduction, even slight, in renal function has a negative effect on the patient's prognosis. In this sense, the diagnosis and timely treatment of AKI can improve the patient's clinical course and prognosis.

Unlike other vital parameters such as blood pressure, heart rate and oxygen concentration in the blood, physicians currently do not have reliable real-time monitoring systems available for a critical parameter such as the diuresis level.

To date, the monitoring is carried out manually by ward nurses who periodically (approximately every six hours) check the diuresis level through the graduated marks on the urine collection bags.

Examples of this type of monitoring are given in E. Macedo et al., Preventing Acute Kidney Injury, Critical Care Clinics, Volume 31, Issue 4, pages 773-784.

The main issues related to this type of procedure concern the poor accuracy of the measurement and the lack of an automated data collection system; this implies the failure to recognize anomalies in the course of the patient's diuresis by the attending physician, with the consequent underestimation of even moderate signals that could predict serious clinical consequences.

The study “National Confidential Inquiry into Patient Outcome and Death”, published in 2009, on the management of patients who developed acute kidney failure during hospitalization (see London, UK, https://www.ncepod.org.uk/CommonThemes.pdf), in fact estimated that 43% of patients received a late diagnosis of the onset of AKI and that in 54% of the cases the risk of developing AKI was underestimated by the attending physician.

Several patent documents report systems that aim to monitor the patient's diuresis for the patient's best management in a hospital environment.

The document WO 2008/059483 A3 describes a system for monitoring body fluids based on optical measurements.

The drawbacks of this solution result to be the difficulty in maintaining sterility of the measurement system, the technical complexity of the solution and the consequent problems of technical nature, the lack of data connectivity and processing for predicting the patient's future state of health and the overall dimensions of the device.

The document EP 3282948 A1 describes a system for monitoring the renal function comprising a device for monitoring urine and an algorithm associated with it for calculating the risk of AKI. The unsolved problems of this solution are mainly of two types.

Firstly, the overall dimensions of the monitoring device are considerable, due to the presence of a platform for calculating the weight of the urine bag, which does not allow the use of the system without hindering the work of doctors and healthcare professionals around the hospital bed.

The second problem is related to the type of output coming out of the AKI risk calculation system proposed in the aforementioned solution.

For diagnostic assistance systems, such as the one proposed in the aforementioned solution, the degree of understanding and reception by the recipient of the information, in this case, the doctor, of the information provided by the system, plays a key role.

The output proposed in the aforementioned solution consists of a “risk score” that defines the probability of occurrence of AKI for the corresponding patient. Numerous studies (eg, K. B. Kashani “Automated acute kidney injury alerts” J. Kidney Intern. September 2018, Volume 94, Issue 3, pages 484-490; K. B. Kashani, E. A. Burdmann, L. Seong Hooi, D. Khullar, A. Bagga, R. Chakravarthi, R. Mehta, “Acute Kidney Injury Risk Assessment: Differences and Similarities Between Resource-Limited and Resource-Rich Countries” Kidney International Reports, 2017, Volume 2, Issue 4, pages 519-529) have shown how this information is not understood and poorly implemented by doctors and, consequently, is not integrated into clinical practice, losing its impact on improving patients' health.

The document WO 2017/149272 A1 describes a system for monitoring body fluids, namely urine, based on a load cell.

The drawbacks of this solution result to be the lack of a system for predicting the onset of renal insufficiency and the overall dimensions of the device.

More generally, document U.S. Pat. No. 4,922,922 A describes a system for monitoring body fluids released by the patient during surgical operations.

The problems of this solution reside in the lack of a system for predicting the future trend of body fluids and in the lack of integration of the quantity of body fluids lost with other vital parameters of the patient, in order to determine his/her state of health.

A system and method capable of monitoring and predicting the diuresis and, more generally, biological fluid, would satisfy the requirements of numerous applications such as, for example, the assessment of the risk of kidney failure and, more generally, the evaluation the state of health of a patient.

The present invention aims to meet the aforementioned requirements.

In particular, the present invention intends to solve the technical problem of how to recognize early the onset of acute kidney injury (AKI).

Moreover, the present invention intends to solve the technical problem of how to improve the quality of management of kidney failure in a hospital environment, consequently reducing the relative mortality rate and the number of ordinary hospitalization days.

Moreover, the present invention intends to solve the technical problem of how to provide the healthcare professionals with the clinical information necessary to correctly manage the onset of acute kidney injury (AKI) by means of a continuous monitoring system, predicting in real-time the trend of diuresis of the patient and assessing in real-time the risk of developing AKI in the future.

Moreover, the present invention intends to solve the technical problem of how to reduce the size of diuresis monitoring devices.

Moreover, the present invention intends to solve the technical problem of how to early recognize the alteration of a patient's state of health.

Moreover, the present invention intends to solve the technical problem of how to improve the quality of management of alterations in the state of health of patients in a hospital environment, consequently reducing the relative mortality rate and the number of ordinary hospitalization days.

Moreover, the present invention intends to solve the technical problem of how to provide the medical personnel with the clinical information necessary to correctly manage the onset of alterations in the state of health of the patient by means of a continuous monitoring system, of real-time forecasting of the trend of biological fluid of the patient and real-time assessment of the risk of worsening health conditions in the future.

In summary, therefore, up to date, to the Applicant's knowledge, no solutions are known which allow to monitor and predict diuresis and, more generally, biological fluid, for the assessment of the risk of kidney failure and, more generally, for the evaluation of a patient's state of health. Therefore, the Applicant, with the systems and methods according to the present invention, intends to remedy this lack.

### SUMMARY

The object of the present invention is to overcome the drawbacks of the known art linked to the impossibility of monitoring and predicting diuresis for the assessment of the risk of kidney failure.

More generally, it is an object of the present invention to overcome the drawbacks of the known art linked to the impossibility of monitoring and predicting the flow of biological fluid for the evaluation of a patient's state of health.

Such objectives are achieved with the systems and methods according to the present invention which, advantageously and thanks to the detection, recording and processing over time of a vital parameter of the patient, specifically of the diuresis, allow to monitor and predict the flow of biological fluid of a patient, in particular diuresis, for the evaluation of the patient's state of health, in particular the risk of onset of AKI.

The systems and methods according to the present invention combine for the first time, to the Applicant's knowledge, the detection of the weight of samples taken from a patient over time, the recording and processing of such weight data useful to identify trends over time useful to the early diagnosis of the onset of diseases, in particular the onset of AKI.

Specifically, the aforementioned and other objects and advantages of the invention, as will appear from the following description, are achieved with a diuresis monitoring system for predicting the risk of kidney failure of a patient according to claim 1.

Preferred embodiments and variants of the diuresis monitoring system according to the present invention form the subject matter of the dependent claims 2 to 5.

Another independent aspect of the present invention relates to a diuresis monitoring method for predicting the risk of kidney failure of a patient and constitutes the subject matter of claim 6.

Preferred embodiments and variants of the diuresis monitoring method according to the present invention form the subject matter of the dependent claims 7 and 8.

Another independent aspect of the present invention relates to a diuresis prediction method for calculating the risk level of acute kidney failure of a patient and constitutes the subject matter of claim 9.

Preferred embodiments and variants of the diuresis prediction method to the present invention form the subject matter of the dependent claims 10 to 12.

Another independent aspect of the present invention relates to a monitoring method of biological fluid for predicting the state of health of a patient and constitutes the subject matter of claim 13.

Preferred embodiments and variants of a monitoring method of biological fluid according to the present invention form the subject matter of the dependent claims 14 and 15.

Another independent aspect of the present invention relates to a monitoring method of biological fluid for predicting the state of health of a patient and constitutes the subject matter of claim 16.

Preferred embodiments and variants of a monitoring method of biological fluid according to the present invention form the subject matter of the dependent claims 17 to 19.

Another independent aspect of the present invention relates to the prediction method of biological fluid for calculation of the level of the state of health of a patient and constitutes the subject matter of claim 20.

Preferred embodiments and variants of a predicting method of biological fluid according to the present invention form the subject matter of the dependent claims 21 to 24.

It is understood that all the appended claims form an integral part of the present description and that each of the technical characteristics claimed therein is possibly independent and can be used autonomously with respect to the other aspects of the invention.

It will be immediately apparent that countless modifications could be made to what described (for example, related to shape, sizes, arrangements and parts with equivalent functionalities) without departing from the scope of protection of the invention as claimed in the appended claims.

Advantageously, the technical solution according to the present invention, which provides systems and methods for monitoring and predicting the progress of a vital parameter of a patient, allows:
to overcome the clinical procedures currently in use thanks to the introduction, in the future clinical practice related to the management of diuresis of catheterized patients, of innovative systems and methods;
through the continuous and automatic monitoring of the diuresis or, more generally, of biological fluid of a patient, and the automatic and instantaneous evaluation (through instantaneous comparison with parameters and thresholds obtained from international guidelines already shared and accepted by the clinical medical community) of the severity stage of acute kidney failure or, more generally, of alterations of the state of health, to allow a more accurate and early diagnosis of problems and, in particular of the onset of AKI, with respect to the current clinical standard;
through the instant and automatic alert system of the attending physician in the event of an increase in the risk level of AKI onset or, more generally, of abnormal alteration of a vital parameter, to start a more timely intervention and therapy of the syndrome in progress, with the possibility of avoiding the worsening of the clinical picture and the consequent complications;
by sending and saving the collected data in real time, to analyse ex-post the diuresis trends over time, or of any biological fluid of the patient, with the aim of developing a predictive model capable to diagnose early and accurately the onset of AKI or another pathology;
to improve significantly the clinical treatment of acute kidney injury in hospitalized and catheterized patients, allowing early diagnosis and clinical intervention;
to improve the quality of life of catheterized patients;
to reduce the health expenditure related to the management of acute kidney failure, reducing the number of hospitalization days in intensive care and the number of readmissions to the hospital;
to allow the development of predictive models on the course of the disease according to the different therapeutic approaches;
to allow the development of a monitoring system consisting of two different devices; the first one, minimally invasive and capable of not hindering the medical activity around the hospital bed and the second one, most cumbersome in an area placed such as not to hinder;
to allow the development of a predictive algorithm capable to predict the future trend of biological fluids including the diuresis of the catheterized patient, and from this calculate the risk level of onset of AKI.

Further advantageous characteristics will become more apparent from the following description of preferred, but not exclusive embodiments provided purely by way of example and not of limitation.

### BRIEF DESCRIPTION OF THE DRAWINGS

The present invention will be described hereinafter by way of some preferred embodiments, provided by way of example and not of limitation, with reference to the accompanying drawings. These drawings illustrate different aspects and examples of the present invention and, where appropriate, similar structures, components, materials and/or elements in different figures are denoted by similar reference numerals.

FIG. 1 is a schematic representation of the diuresis monitoring system for predicting the risk of kidney failure of a patient according to the present invention;

FIG. 2 is a flow diagram of the diuresis monitoring system for predicting the risk of kidney failure of a patient according to the present invention;

FIG. 3 is a schematic representation of the monitoring system of biological fluid for predicting the state of health of a patient according to the present invention;

FIG. 4 is a flow diagram of the monitoring method of biological fluid for predicting the state of health of a patient according to the present invention;

FIG. 5 is a schematic representation which illustrates the set of elaborations performed by the second algorithm of the device of the diuresis monitoring system for the prediction of the risk of kidney failure of a patient according to the present invention; and

FIG. 6 is a schematic representation which illustrates the set of elaborations performed by the second algorithm of the device of the monitoring system of biological fluid for the prediction of the state of health of a patient according to the present invention.

### DETAILED DESCRIPTION OF THE EMBODIMENTS

While the invention is susceptible to various modifications and alternative implementations, some preferred embodiments are shown in the drawings and will be described in detail hereinbelow.

It should be understood, however, that there is no intention to limit the invention to the specific embodiments illustrated, but, on the contrary, the invention is intended to cover all modifications, alternative implementations, and equivalents which fall within the scope of the invention as defined in the claims.

In the following description, therefore, the use of “for example”, “etc.”, “or”, “either” indicates not exclusive alternatives without any limitation, unless otherwise indicated; the use of “also” means “including, but not limited to” unless otherwise indicated; the use of “includes/comprises” means “includes/comprises but not limited to” unless otherwise indicated.

The systems and methods of the present invention are based on the innovative concept of combining the detection of the weight of samples taken from a patient over time, the recording and processing of such weight data to identify trends over time useful to the early diagnosis of the onset of diseases, in particular the onset of AKI.

In summary, the systems and methods of the present invention exploit:
automatic and real-time monitoring of the catheterized patient's diuresis by means of two instruments able to communicate with each other via the Bluetooth network and able to transfer data via the 3G network for telemonitoring the state of health of the patient by the attending physician; advantageously, this allows to reduce the overall dimensions of the equipment in the area surrounding the patient's bed;
algorithms implemented into the two instruments for the determination of the stage of progress of acute kidney failure and the related risk levels; and
processes for collecting, processing and transmitting data to the attending physician.

The present invention has, as its primary object, the early diagnosis of acute kidney failure in hospitalized catheterized patients and, in general, the early diagnosis of a worsening of their health.

The systems and methods of the present invention, through the constant monitoring of the patient's diuresis—or another vital parameter—allow to identify automatically and in real time any discrepancies with respect to a physiological diuretic regimen—or a state of health—so as defined by international guidelines; moreover, the systems and methods of the present invention, through remote connections, allow to inform the attending physician of the possible overcoming of the alert threshold and, consequently, to carry out an early diagnosis and a timely therapeutic intervention.

In the present description, the term “biological fluid” means a fluid of human origin comprising, but not limited to urine, blood and other blood products, saliva, mucus, amniotic fluid, peritoneal fluid, lymphatic system fluid, gastric fluid, blood, body fluids in general.

In the present description, the terms “electronic clinical record” and “electronic medical record” mean the set of data collected relating to the patient and relating to his state of health, comprising but not limited to blood creatinine level, arterial pressure, heart rate and electrocardiogram, body temperature, oxygen saturation, respiratory rate, patient's weight, amounts of fluids administered to the patient and current diseases; in the present description the terms “electronic clinical record” and “electronic medical record” are used without distinction, as synonyms.

In the present description, the term “KDIGO guidelines” means the guidelines for the management of acute kidney failure described in the document “The 2012 Kidney Disease: Improving Global Outcomes (KDIGO) Clinical Practice Guideline for Acute Kidney Injury (AKI)” (source: Web site https://kdigo.org/guidelines/acute-kidney-injury/; access date: Jan. 28, 2019)

In the present description, the term “RIFLE guidelines” means the guidelines for the management of acute kidney failure described in “Bellomo R., Ronco C., Kellum J. A., et al., Acute renal failure—definition, outcome measures, animal models, fluid therapy and information technology needs: the Second International Consensus Conference of the Acute Dialysis Quality Initiative (ADQI) Group. Crit Care 2004; 8:R204-212”.

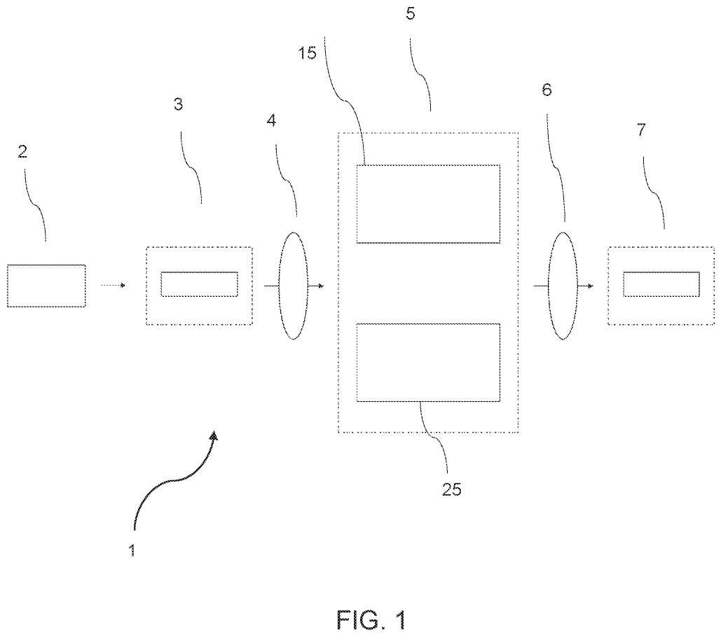

With reference to FIG. 1, which illustrates the preferred embodiment of the present invention, it is observed that the diuresis monitoring system 1 for predicting the risk of kidney failure of a patient, comprises:
a urine container 2;
a weight meter 3 of the urine container 2;
a device 5 comprising a first algorithm 15 for recording, storing, comparing and processing the measurements of the urine container 2 and a second algorithm 25 for predicting the future measurements of the urine container 2 and the level of kidney failure risk associated with them;
a videoterminal 7 for displaying the outputs of the first algorithm 15 and the second algorithm 25 present in the device 5;
a first wireless system 4 for connecting the weight meter 3 and the device 5; and
a second wireless system 6 for connecting the device 5 and the videoterminal 7.

Preferably, the urine container 2 is a sterile bag.

Preferably, the weight meter 3 is a load cell.

Preferably,
the first algorithm 15 comprises a mathematical model for the analysis of the data obtained through the weight meter 3 in order to correlate each weight measurement with the time instant in which it was performed and to calculate a rate of hourly urinary production normalized on the weight of the patient (weight/hour/patient's weight); subsequently, such normalized hourly urinary production rate is compared with the hourly production rate thresholds defined by the KDIGO and RIFLE guidelines for the definition of stages of acute kidney failure (AKI);
the second algorithm 25 comprises:
   an adaptive mathematical model H-25 having as input at least the present value and the past values of diuresis as calculated by the first algorithm 15 and, if relevant, the present value and the past values extracted from the patient's electronic medical record 35 and having as output the predictions of future container weight measurements UO(t)\hat{ };
   a mathematical model for comparing e(t)\hat{ } of the predictions UO(t)\hat{ } with the corresponding values observed in real time UO(t);
   a mathematical model for correcting the calculation performed by the adaptive mathematical model H-25 on the basis of the result of the comparison e(t)\hat{ }; and
   a mathematical model M-25 having as input the output of the adaptive mathematical model H-25, the present value and the past values of the weight measurements of the urine container 2 and the physiological parameters present in the patient's electronic medical record 35, and having as output the risk level
      ranging from 1 to 10—of developing an acute kidney failure within 24/48 hours after the last weight measurement R(t)\hat{ } of the urine container.

Preferably, the adaptive mathematical model H-25 comprises linear and non-linear regression models and machine-learning models, preferably artificial neural networks.

Preferably, the mathematical model M-25 comprises regression models with a variable dichotomous response, more preferably logit and probit models, machine-learning models, preferably classification models, artificial neural networks and SVM models.

Preferably, the present value and the past values extracted from the patient's electronic medical records 35 comprise blood creatinine level, arterial pressure, heart rate and electrocardiogram, body temperature, oxygen saturation, respiratory rate, patient's weight, amount of fluids administered to the patient and current diseases.

In an exemplary and non-limiting embodiment, the diuresis monitoring system 1 comprises a hardware component and a software component.

The hardware component comprises a weight meter 3 and a device 5.

The weight meter 3 has the task of measuring the amount of diuresis present inside the urine bag 2 used by the catheterized patient in a hospital environment; such measurement is carried out by calculating the weight of the urine bag 2.

The data thus collected is then transferred via Bluetooth connection to the device 5.

The hardware components of the weight meter 3 are:
a battery-powered microcontroller equipped with a Low-Energy-Bluetooth (BLE) connection used to manage the weight measurement of the urine bag and to send the data via Bluetooth connection to the weight meter 3;
a load cell used for measuring the weight of the urine bag;
a 24-bit analogue-to-digital converter (ADC) used to amplify and convert the measurement signal generated by the load cell relative to the weight of the urine bag; the data thus processed is transferred by cable to the microcontroller described above;
a casing used to contain all the hardware components necessary for the operation of the weight meter 3;
a chain and hook used for connecting the weight measurement sensor and urine bag; the bag results then suspended and hooked to the hook.

The weight meter 3 is attached to the bed structure of the patient in intensive care and will be small in size so as not to hinder the daily work of the medical staff who operates near the patient's bedside and that often requires immediate intervention, from which the survival of the patient may depend.

It is wanted to point out that no type of invasive operation is carried out either on the urine-containing bag, nor on the patient's urinary flow, nor is it necessary to use bags of particular type or manufacture for the use of the weight meter 3.

The device 5 has the task of
receiving the data transmitted by the weight meter 3 via Bluetooth connection related to the weight of the urine bag,
analysing and processing the data received,
transmitting the processed data to a smartphone application via 3G connection for later viewing by the attending physician,
transmitting the raw data to an electronic database for storing the collected data via a 3G connection,
allowing raw and processed data to be displayed to the department nurse,
allowing information about the patient being monitored to be entered.

The hardware components of device 5 are:
a microcontroller powered by a power outlet, equipped with a 3G and Bluetooth connection, used to manage the reception of data transmitted by the weight meter 3 via Bluetooth connection, the processing of the aforementioned data and the display of the raw and processed data on a capacitive touchscreen; and
a capacitive touchscreen used for displaying raw and processed data by the microcontroller and for entering the patient information by the nurse on duty on the ward.

The software component includes software for the weight meter 3 and software for device 5. The software of the weight meter 3, implemented inside the corresponding microcontroller, has the task of
managing the data collection of the load cell, measuring the weight of the urine bag every 5 minutes,
transmitting the data collected into the device 5 via Bluetooth connection.

Moreover, such software is optimized to minimize the power consumption of the weight meter 3, which will allow it to be powered by batteries.

The software of the device 5, implemented inside the corresponding microcontroller, has the task of
managing the reception via BLE connection of the data transmitted by the weight meter 3 to the device 5;
processing the data received; such processing has the purpose of determining the risk of onset of acute kidney failure or acute kidney injury or kidney injury, associating each patient with a level of risk; such risk is calculated by comparing the diuresis of the last 24 hours of the monitored patient (obtained by measuring the weight of the urine bag over time) with the thresholds determined by the international guidelines “KDIGO Clinical Practice Guideline for Acute Kidney Injury” (March 2012) for the definition of the progression stage of acute renal injury (AKI); moreover, a machine-learning algorithm will be implemented with the aim of determining and implementing a predictive model of acute renal injury;
managing the display of the processed data and the risk levels calculated using the capacitive touchscreen present in device 5;
managing the input of sensitive data relating to the monitored patient such as the code assigned to each patient, the weight in kg and the age, by the health personnel through the touchscreen display;
managing the transmission of processed data and risk levels calculated through 3G connection to the on-line database and the smartphone application.

In summary, the diuresis monitoring system 1 according to the present invention, for hospitalized catheterized patients, substantially comprises:
data collection: continuous measurement of the weight of the hospitalized patient's urine bag
elaboration of the data collected: calculation of the patient's diuresis over time in mL/hr/kg (urine output) which provides for the comparison of such urine output with the thresholds described by the international guidelines KDIGO for the definition of the stage of progress of the kidney failure associated with a certain level of risk, and the processing of the patient's diuresis with machine-learning algorithms for the determination and implementation of a predictive model of the onset of acute kidney failure leading to the determination of accurate risk levels of onset of acute kidney failure;
communication of processed data and patient risk levels to the attending physician; such communication, which has the purpose of allowing the doctor to know in real time the diuresis of the patient and the risk levels associated with it, and consequently to have the possibility to promptly intervene on the patient's health condition, by sending the data documents and risk levels to a smartphone application owned by the attending physician.

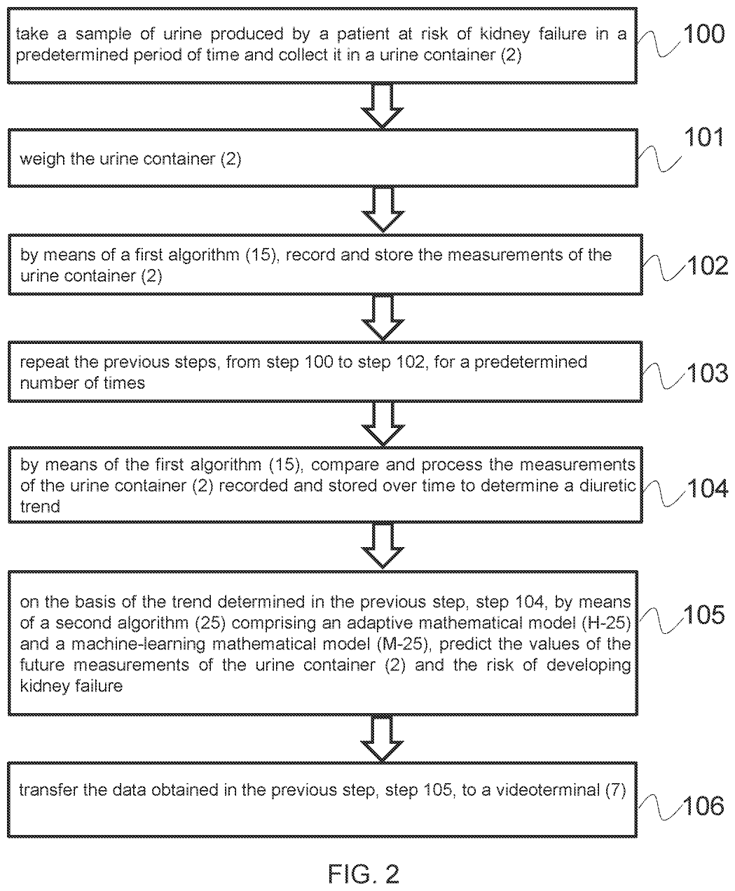

Furthermore, with reference to FIG. 2 that illustrates the preferred embodiment of the present invention, a diuresis monitoring method for predicting the risk of kidney failure of a patient constitutes an independent aspect usable independently with respect to the other aspects of the invention and comprises the following steps:
taking a sample of urine produced by the patient at risk of kidney failure in a predetermined period of time and collecting it in a urine container 2 (step 100);
weighing the urine container 2 (step 101);
by means of a first algorithm 15, recording and storing the measurements of the urine container 2 (step 102);
repeating the previous steps, from step 100 to step 102, for a predetermined number of times (step 103);
by means of the first algorithm 15, comparing and processing the measurements of the urine container 2 and stored over time to determine a diuretic trend (step 104);
on the basis of the trend determined in the previous step, step 104, by means of a second algorithm 25 comprising an adaptive mathematical model H-25 and a machine-learning mathematical model M-25, predicting the values of future measurements of the urine container 2 and the risk of developing kidney failure (step 105);
transferring the data obtained in the previous step, step 105, to a videoterminal 7 (step 106).

Preferably,
the first algorithm 15 comprises a mathematical model for the analysis of the data obtained through the weight meter 3 in order to correlate each weight measurement with the time instant in which it was performed and to calculate a rate of hourly urinary production normalized on the weight of the patient (weight/hour/patient's weight); subsequently, this normalized hourly urinary production rate is compared with the hourly production rate thresholds defined by the KDIGO and RIFLE guidelines for the definition of stages of acute kidney failure AKI;
the second algorithm 25 comprises:
   an adaptive mathematical model H-25 having as input at least the present value and the past values of diuresis as calculated by the first algorithm 15 and, if relevant, the present value and the past values extracted from the patient's electronic medical record 35 and having as output the predictions of future container weight measurements UO(t)\hat{ };
   a mathematical model for comparing e(t)\hat{ } the predictions UO(t)\hat{ } with the corresponding values observed in real time UO(t);
   a mathematical model for correcting the calculation performed by the adaptive mathematical model H-25 on the basis of the comparison result e(t)\hat{ }; and
   a mathematical model M-25 having as input the output of the adaptive mathematical model H-25, the present value and the past values of the weight measurements of the urine container 2 and the physiological parameters present in the patient's electronic medical record 35, and having as output the risk level
   ranging from 1 to 10—to develop an acute kidney failure within 24/48 hours after the last weight measurement of the urine container R(t)\hat{ }.

Preferably,
the predetermined period of time referred to at step 100 ranges from 30 seconds to 10 minutes, preferably it is equal to 5 minutes; and
the predetermined number of times referred to at step 103 ranges from 1 to 100, preferably it is equal to 50.

Furthermore, a prediction method of diuresis UO(t) for calculating the risk level of acute kidney failure of a patient constitutes an independent aspect that can be used autonomously with respect to the other aspects of the invention and comprises the following steps:
by means of an adaptive mathematical model H-25, calculating the trend of the patient's diuresis that considers
   at least the present value and the past values of diuresis as recorded and processed by a device 5, and
   optionally, if relevant, the present value and the past values extracted from the patient's electronic clinical record related to blood creatinine level, arterial pressure, heart rate and electrocardiogram, body temperature, oxygen saturation, respiratory rate, patient's weight, amounts of fluids administered to the patient and current diseases (step 300);
comparing the expected value UO(t)\hat{ }, i.e. of the calculation output referred to at step 300, with the corresponding values observed in real time UO(t) (step 301);
correcting the calculation referred to at step 300 on the basis of the comparison referred to at step 301 (step 302);
comparing the predicted values UO(t)\hat{ }, UO(t+1)\hat{ }, UO(t+2)\hat{ }, i.e. the calculation outputs referred to at step 300, with the thresholds indicated in the KDIGO and AKIN guidelines for the diagnosis of acute kidney failure (step 303);
assigning a risk level—ranging from 1 to 10—of developing acute kidney failure based on the comparison referred to at step 303 (step 304);
by means of a machine-learning mathematical model M-25, calculating the risk factor of kidney failure in the future instants that considers:
   at least the present value (UO(t) of the past values and the values predicted by the adaptive mathematical model H-25 of diuresis and
   optionally, if relevant, the present value and the past values extracted from the patient's electronic clinical record related to blood creatinine level, arterial pressure, heart rate and electrocardiogram, body temperature, oxygen saturation, respiratory rate, patient's weight, amounts of fluids administered to the patient and current diseases (step 305).

Preferably, the adaptive mathematical model H-25 is a model whose calibration algorithm considers the available additional information relevant to the patient provided in real time, for example through the use of Bayesian estimators.

Preferably, the predicted values UO(t)\hat{ }, UO(t+1)\hat{ }, Uo(t+2)\hat{ } referred to at step 303 are relevant to corresponding time instants t, t+1, t+2 increased so that each increment is a temporal value ranging from 5 minutes to 6 hours.

Preferably, the machine-learning mathematical model M-25 is selected from regression models with variable dichotomous response (including logit and probit models) and machine-learning models (including classification models, artificial neural networks and SVM models).

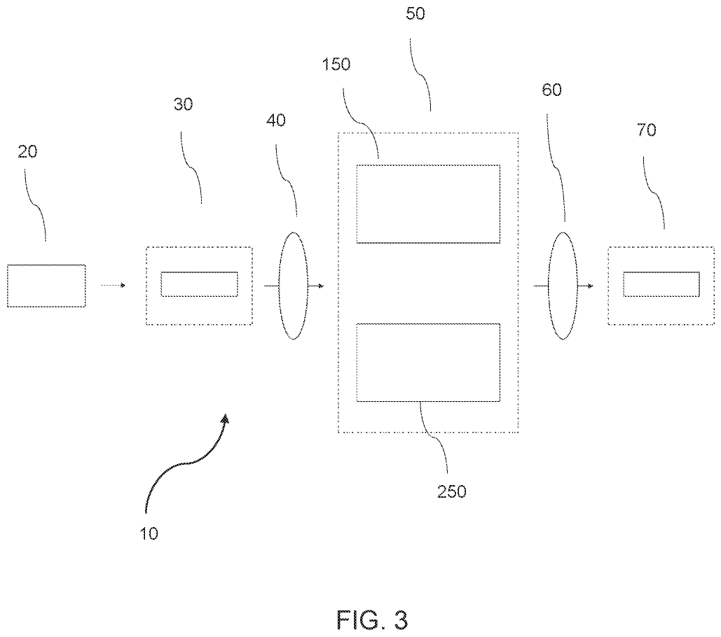

With reference to FIG. 3, which illustrates a general embodiment of the present invention, a monitoring method of biological fluid 10 for predicting the state of health of a patient constitutes an independent aspect that can be used autonomously with respect to the other aspects of the invention and comprises:
a container of biological fluid 20;
a weight meter 30 the biological fluid container 20;
a device 50 comprising a first algorithm 150 for recording, storing, comparing and processing among them the measurements of the biological fluid container 20 and a second algorithm 250 for predicting the future measurements of the biological fluid container 20 and the patient's state of health associated with them;
a videoterminal 70 for displaying the outputs of the first algorithm 150 and the second algorithm 250 present in the device 50;
a first wireless system 40 for connecting the weight meter 30 and the device 50; and
a second wireless system 60 for connecting the device 50 and the videoterminal 70.

Preferably, the biological fluid is selected among peritoneal fluid, lymphatic fluid, urine, blood, amniotic fluid and saliva.

Preferably, the biological fluid container 10 generally is a sterile bag.

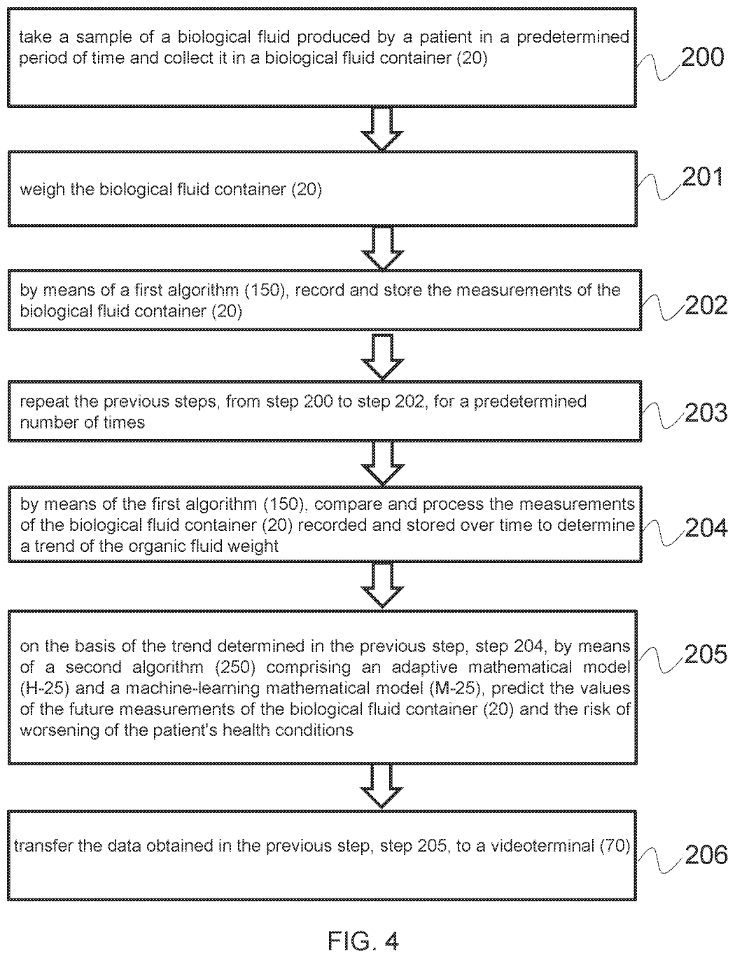

Furthermore, with reference to FIG. 4, which illustrates a general embodiment of the present invention, a monitoring method of biological fluid for predicting the state of health of a patient constitutes an independent aspect that can be used autonomously with respect to the other aspects of the invention and comprises the following steps:
taking a sample of biological fluid produced by the patient in a predetermined period of time and collecting it in biological fluid container 20 (step 200);
weighing the biological fluid container 20 (step 201);
by means of a first algorithm 150, recording and storing the measurements of the biological fluid container 20 (step 202);
repeating the previous steps, from step 200 to step 202, for a predetermined number of times (step 203);
by means of the first algorithm 150, comparing and processing the measurements of the biological fluid container 20 and stored over time to determine a trend of the organic fluid weight (step 204);
on the basis of the trend determined in the previous step, step 204, by means of a second algorithm 250 comprising an adaptive mathematical model H-250 and a machine-learning mathematical model M-250, predicting the values of the future measurements of the biological fluid container 20 and the risk of worsening of the patient's health conditions (step 205); and
transferring the data obtained in the previous step, step 205, to a videoterminal 70 (step 206).

Preferably, the biological fluid is selected from peritoneal fluid, lymphatic fluid, urine, blood, amniotic fluid and saliva.

Preferably,
the first algorithm 150 comprises a mathematical model for the analysis of the data obtained through the weight meter 30 in order to correlate each weight measurement with the time instant in which it was performed and to calculate a rate hourly production of biological fluid normalized on the weight of the patient (weight/hour/patient's weight);
the second algorithm 250 comprises:
   an adaptive mathematical model H-250 having as input at least the present value and the past values of the biological fluid flow as calculated by the first algorithm 150 and, if relevant, the present value and the past values extracted from the patient's electronic medical record 350 and having as output the predictions of future container weight measurements UO(t)\hat{ };
   a mathematical model for comparing e(t)\hat{ } the predictions UO(t)\hat{ } with the corresponding values observed in real time UO(t);
   a mathematical model for correcting the calculation performed by the adaptive mathematical model H-25 on the basis of the comparison result e(t)\hat{ }; and
   a mathematical model M-250 having as input the output of the adaptive mathematical model H-250, the present value and the past values of the weight measurements of the biological fluid container 20 and the physiological parameters present in the patient's electronic medical record 350, and having as output the risk level—ranging from 1 to 10—of worsening of the patient's health in 24/48 hours after the last weight measurement of the biological fluid container R(t)\hat{ }.

Preferably
the predetermined period of time referred to at step 200 ranges from 30 seconds to 10 minutes, preferably it is equal to 5 minutes; and
the predetermined number of times referred to at step 203 ranges from 1 to 100, preferably it is equal to 50.

Furthermore, a prediction method of the biological fluid flow for calculating the level of a patient's state of health constitutes an independent aspect autonomously usable with respect to the other aspects of the invention and comprises the following steps:
by means of an adaptive mathematical model H-250, calculating the trend of the patient's biological fluid that considers
   at least the present value and the past values of the biological fluid as recorded and processed by a device 50 and
   optionally, if relevant, the present value and the past values extracted from the patient's electronic clinical record related to blood creatinine level, arterial pressure, heart rate and electrocardiogram, body temperature, oxygen saturation, respiratory rate, patient's weight, amounts of fluids administered to the patient and current diseases (step 400);
comparing the expected value UO(t)\hat{ }, i.e. the calculation output referred to at step 400, with the corresponding values observed in real time UO(t) (step 401);
correcting the calculation referred to at step 400 on the basis of the comparison referred to at step 401 (step 402);
by means of a machine-learning mathematical model M-250, calculating the level of state of health in the future instants that considers.
   at least the present value UO(t), the past values and the values predicted by the adaptive mathematical model H-250 of the biological fluid and
   optionally, if relevant, the present value and the past values extracted from the patient's electronic clinical record related to blood creatinine level, arterial pressure, heart rate and electrocardiogram, body temperature, oxygen saturation, respiratory rate, patient's weight, amounts of fluids administered to the patient and current diseases (step 405).

Preferably, the adaptive mathematical model H-250 is a model whose calibration algorithm considers the available additional information relevant to the patient provided in real time, for example through the use of Bayesian estimators.

Preferably, the predicted values UO(t)\hat{ }, UO(t+1)\hat{ }, Uo(t+2)\hat{ } referred to at step 403 are relevant to corresponding time instants t, t+1, t+2 increased so that each increment is a temporal value ranging from 5 minutes to 6 hours.

Preferably, the machine-learning mathematical model M-250 is selected from regression models with variable dichotomous response (including logit and probit models) and machine-learning models (including classification models, artificial neural networks and SVM models).

Preferably, the biological fluid is selected from peritoneal fluid, lymphatic fluid, urine, blood, amniotic fluid and saliva.

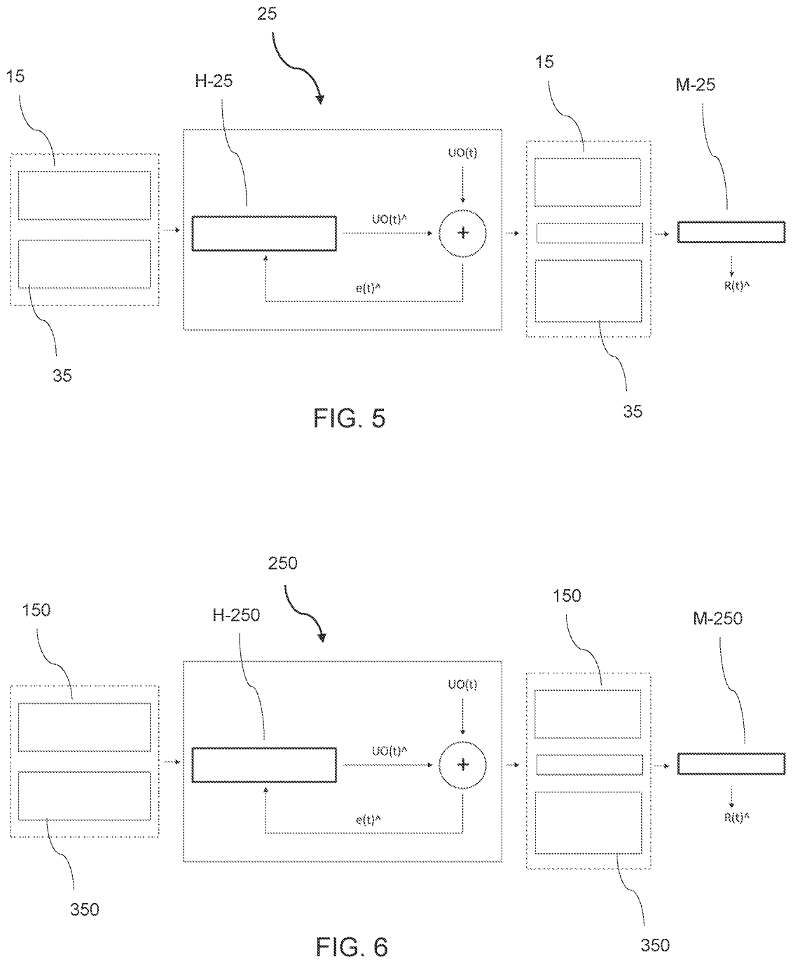

With reference to FIG. 5, which represents the set of elaborations performed by algorithm 25 of the device 5 of the diuresis monitoring system 1 for the prediction of the risk of kidney failure of a patient according to the present invention, the algorithm 25 comprises:
an adaptive mathematical model H-25 having as input at least the present value and the past values of diuresis as calculated by the first algorithm 15 and, if relevant, the present value and the past values extracted from the patient's electronic medical record 35 and having as output the predictions of future container weight measurements UO(t)\hat{ };
a mathematical model for comparing e(t)\hat{ } the predictions UO(t)\hat{ } with the corresponding values observed in real time UO(t);
a mathematical model for correcting the calculation performed by the adaptive mathematical model H-25 on the basis of the comparison result e(t)\hat{ }; and
a mathematical model M-25 having as input the output of the adaptive mathematical model H-25, the present value and the past values of the weight measurements of the urine container 2 and the physiological parameters present in the patient's electronic medical record 35, and having as output the risk level—ranging from 1 to 10—to develop an acute kidney failure within 24/48 hours after the last weight measurement of the urine container R(t)\hat{ }.

With reference to FIG. 6, which represents the set of elaborations performed by algorithm 250 of the device 50 of biological fluid monitoring system 10 for the prediction of the state of health of a patient according to the present invention, the algorithm 250 comprises:
an adaptive mathematical model H-250 having as input at least the present value and the past values of the biological fluid flow as calculated by the first algorithm 150 and, if relevant, the present value and the past values extracted from the patient's electronic medical record 350 and having as output the predictions of future container weight measurements UO(t)\hat{ };
a mathematical model for comparing e(t)\hat{ } the predictions UO(t)\hat{ } with the corresponding values observed in real time UO(t);
a mathematical model for correcting the calculation performed by the adaptive mathematical model H-250 on the basis of the comparison result e(t)\hat{ }; and
a mathematical model H-250 having as input the output of the adaptive mathematical model H-250, the present value and the past values of the weight measurements of the biological fluid container 20 and the physiological parameters present in the patient's electronic medical record 350, and having as output the risk level—ranging from 1 to 10—of worsening of the patient's health in 24/48 hours after the last weight measurement of the urine container R(t)\hat{ }.

The systems and methods according to the present invention are described below in greater detail with reference to the following Examples, which have been developed on the basis of experimental data and which are intended as illustrative, but not limitating, of the present invention.

### Example 1

The weight meter of the urine bag, having the functionalities described above, can have, for example, a L×W×H size of 5×5×5 cm; it can be equipped with a rechargeable 3.7 V and 500 mAh lithium-ion battery power supply with a long-life having a 29×36×4.75 mm size; a miniaturized battery charger having a size of 35×33×7 mm: a STM32L476JG processor mounted on the SensorTile module for the control and the management of the data collected by the sensor and their sending via low-power Bluetooth connection to the urine container; a miniaturized load cell weight sensor, specifically S215-012, with a 5.4 kg capacity, a 28.7×5.99×5.99 mm size and a ±1 g accuracy; a 24-bit analogue-to-digital converter (ADC) for load cells, specifically the HX711 model, having a 31×22 mm size; of an enclosure having a 5×5×5 cm size made of ABS and IP68 watertight.

The device, denoted with the reference number 5 and having the functionalities described above, can have, for example, overall dimensions of 20×10×10 cm; it can be equipped with a power supply with a socket; a 3.5″ touchscreen for data input by the user; a Rasperry Pi3 b plus microcontroller for the control and management of data received via low-energy Bluetooth connection, and which implements the previously described algorithms therein; the Raspberry pi 3G 4G LTE base shield v2 electronic card, used to connect the urine container to the mobile network.

The systems and methods according to the present invention are compared with known solutions, as described below.

The results of the comparison between the present invention and the known solutions are summarized in the Table below.

TABLE

| | Manual | WO2008/ | | WO2017/ | Present |
|---|---|---|---|---|---|
| | Monitoring | 059483A3 | EP3282948A1 | 149272A1 | invention |
| Diuresis monitoring | NO | YES | YES | YES | YES |
| for short intervals | | | | | |
| (<=10 minutes) | | | | | |
| Overall dimensions | YES | NO | NO | NO | YES |
| minimization | | | | | |
| Prediction of the | NO | NO | NO | NO | YES |
| diuresis/biological | | | | | |
| fluid future trend, | | | | | |
| and associated with | | | | | |
| AKI/acute pathology risk | | | | | |

The aforementioned Table shows the known solutions compared with the present invention; in particular, the main technical features present in the present invention and not present in the previous solutions are highlighted.

The present invention, in the preferred embodiment, represents an innovative system for measuring and analysing the level of diuresis of catheterized patients, with the aim of monitoring the trend of this parameter in order to allow timely therapeutic intervention and obtaining a key indicator for identifying potential critical situations.

The innovative value of the system and methods described is represented above all by the automation of the activity of continuous detection and supervision of the patient's level of diuresis, a parameter that is currently visually verified in an inaccurate way and at prolonged time intervals.

The system allows the instant data collection and continuously calculates the patient's diuresis level, sending this information to a database and making it usable in the future and easily analysable by the attending physician; furthermore, the system is able to analyse the collected data in real time, verify the achievement of therapeutic objectives and overcoming of the diuretic thresholds indicated by the international guidelines for the diagnosis of acute renal injury syndromes AKI.

Secondly, the system allows to optimize the work of the healthcare personnel, since the need to manually supervise if the patients' diuresis level parameters fall or not within the determined and physiological range is eliminated; furthermore, human errors, inevitably common in any repetitive task, such as the supervision of physiological parameters, are limited.

The system is designed to adapt to any type of department and current practices for the management of urethral catheters; consequently, it does not require any further expenditure for the modification of the instrumentation currently in use and can be perfectly integrated to most commercially available catheter bags.

Furthermore, such system does not put the patient's health at risk, as urine does not come into contact with any measuring instrument, guaranteeing sterility and protection from infections.

Given the widespread use of the single technologies used within the systems proposed herein, low production costs are expected.

The systems herein proposed allow, therefore, to combine the strong innovative value deriving from automation, precision and continuity of the collection of information and their ability to actively interact with the attending physician thanks to a system of data connectivity, to an easy-to-use, cost-efficient technology able to suit the environment of use.

As it can be deduced from the foregoing, the innovative technical solution described herein has the following advantageous features:
to overcome the clinical procedures currently in use thanks to the introduction, in the future clinical practice related to the management of diuresis of catheterized patients, of innovative systems and methods;
through the continuous and automatic monitoring of the diuresis or, more generally, of biological fluid of a patient, and the automatic and instantaneous evaluation (through instantaneous comparison with parameters and thresholds obtained from international guidelines already shared and accepted by the clinical medical community) of the severity stage of acute kidney failure or, more generally, of alterations of the state of health, to allow a more accurate and early diagnosis of problems and, in particular of the onset of AKI, with respect to the current clinical standard;
through the instant and automatic alert system to the attending physician in the event of an increase in the risk level of AKI onset or, more generally, of abnormal alteration of a vital parameter, to start a more timely intervention and therapy of the syndrome in progress, with the possibility of avoiding the worsening of the clinical picture and the consequent complications;
by sending and saving the collected data in real time, to analyse ex-post of the diuresis trends over time, or of any biological fluid, of the patient, with the aim of developing a predictive model capable to diagnose in advance and accurately the onset of AKI or another pathology;
to improve significantly the clinical treatment of acute kidney injury in hospitalized catheterized patients, allowing early diagnosis and clinical intervention;
to improve the quality of life of catheterized patients;
to reduce health expenditure related to the management of acute kidney failure, reducing the number of hospitalization days in intensive care and the number of readmissions to the hospital;
to allow the development of predictive models on the course of the disease according to the different therapeutic approaches.

From the description above it is, therefore, apparent how the systems and methods according to the present invention allow achieving the intended objects.

Therefore, it is apparent to a person skilled in the art that it is possible to make modifications and further variants to the solution described with reference to the accompanying figures, without departing from the teaching of the present invention and the scope of protection, as defined by the appended claims.

What is claimed is:

1. A monitoring system of a diuresis for predicting a kidney failure risk of a patient, comprising:
a urine container;
a weight meter of the urine container;
a device comprising a first algorithm for recording, storing, comparing and processing measurements of the urine container and a second algorithm for predicting future measurements of the urine container and a level of the kidney failure risk associated with the future measurements of the urine container;
a videoterminal for displaying outputs of the first algorithm and outputs of the second algorithm present in the device;
a first “wireless” system for connecting the weight meter and the device; and
a second “wireless” system for connecting the device and the videoterminal.

2. The monitoring system according to claim 1, wherein the urine container is a sterile bag.

3. The monitoring system according to claim 2, wherein the weight meter is a load cell.

4. The monitoring system according to claim 3, wherein
the first algorithm comprises a mathematical model for an analysis of data obtained through the weight meter to correlate each weight measurement with a time instant and to calculate a rate of hourly urinary production normalized on a weight of the patient, wherein the analysis was performed in the time instant; subsequently, the rate of hourly urinary production is compared with hourly production rate thresholds defined by KDIGO and RIFLE guidelines for a definition of stages of an acute kidney injury (AKI);
the second algorithm comprises:
an adaptive mathematical model having as an input at least a present value and past values of the diuresis as calculated by the first algorithm and, when relevant, the present value and the past values extracted from an electronic medical record of the patient and having as an output predictions of future container weight measurements;
a first mathematical model for comparing the predictions of the future container weight measurements with corresponding values observed in real time;
a second mathematical model for correcting a calculation performed by the adaptive mathematical model on a basis of a comparison result; and
a third mathematical model having as an input the output of the adaptive mathematical model, a present value and past values of weight measurements of the urine container and physiological parameters present in the electronic medical record of the patient, and having as an output a risk level ranging from 1 to 10 to develop an acute renal failure within 24/48 hours after a last weight measurement of the urine container.

5. The monitoring system according to claim 4, wherein the adaptive mathematical model comprises linear and non-linear regression models and machine learning models, or artificial neural networks, wherein the third mathematical model comprises regression models with a variable dichotomous response, or logit and probit models, the machine learning models, or classification models, the artificial neural networks and support vector machine (SVM) models, and wherein the present value and the past values extracted from electronic medical records of the patient comprise a blood creatinine level, an arterial pressure, a heart rate and an electrocardiogram, a body temperature, an oxygen saturation, a respiratory rate, a weight of the patient, amount of fluids administered to the patient and current diseases.

6. A monitoring method of a diuresis for predicting a kidney failure risk of a patient, comprising the following steps:
step 100) taking a sample of a urine produced by the patient at a risk of kidney failure in a predetermined period of time and collecting the sample of the urine in a urine container;
step 101 weighing the urine container;
step 102) by a first algorithm, recording and storing measurements of the urine container;
step 103) repeating previous steps, from step 100 to step 102, for a predetermined number of times;
step 104) by the first algorithm, comparing and processing the measurements of the urine container recorded and stored over time to determine a diuretic course;
step 105) on a basis of a trend determined in the step 104, by a second algorithm comprising an adaptive mathematical model and a machine learning mathematical model, predicting values of future measurements of the urine container and the risk of kidney failure;
step 106) transferring data obtained in the step 105, to a videoterminal.

7. The monitoring method according to claim 6, wherein:
the first algorithm comprises a mathematical model for an analysis of data obtained through a weight meter to correlate each weight measurement with a time instant and to calculate a rate of hourly urinary production normalized on a weight of the patient, wherein the analysis was performed in the time instant; subsequently, the rate of hourly urinary production is compared with hourly production rate thresholds defined by KDIGO and RIFLE guidelines for a definition of stages of an acute kidney injury (AKI);
the second algorithm comprises:
an adaptive mathematical model having as an input at least a present value and past values of the diuresis as calculated by the first algorithm and, when relevant, the present value and the past values extracted from an electronic medical record of the patient and having as an output predictions of future container weight measurements;
a first mathematical model for comparing the predictions of the future container weight measurements with corresponding values observed in real time;
a second mathematical model for correcting a calculation performed by the adaptive mathematical model on a basis of a comparison result; and
a third mathematical model having as an input an output of the adaptive mathematical model, a present value and past values of weight measurements of the urine container and physiological parameters present in the electronic medical record of the patient, and having as an output a risk level ranging from 1 to 10 to develop an acute renal failure within 24/48 hours after a last weight measurement of the urine container.

8. The monitoring method according to claim 7, wherein:
the predetermined period of time referred to at step 100 ranges from 30 seconds to 10 minutes, or the predetermined period of time is equal to 5 minutes; and
the predetermined number of times referred to at step 103 ranges from 1 to 100, or the predetermined number of times is equal to 50.

9. A prediction method of a diuresis for calculating a risk level of an acute kidney failure of a patient comprising the following steps:
step 300 by an adaptive mathematical model, calculating a trend of the diuresis of the patient considering
at least a present value and past values of the diuresis as recorded and processed by a device and
optionally when relevant, the present value and the past values extracted from an electronic medical record of the patient related to a blood creatinine level, an arterial pressure, a heart rate and an electrocardiogram, a body temperature, an oxygen saturation, a respiratory rate, a weight of the patient, amounts of fluids administered to the patient and current diseases;
step 301) comparing an expected value, wherein a calculation output referred to step 300, with corresponding values observed in real time;
step 302) correcting a calculation referred to at step 300 on a basis of a comparison referred to in step 301;
step 303) comparing predicted value, wherein calculation outputs referred to at step 300, with thresholds indicated in “KDIGO and AKIN Guidelines” for a diagnosis of the acute kidney failure;
step 304) assigning a risk level ranging from 1 to 10 to develop the acute kidney failure based on a comparison referred to at step 303;
step 305) by a machine learning mathematical model, calculating a risk factor of a kidney failure in future instants considering:
at least the present value, the past values and values predicted by the adaptive mathematical model of the diuresis and
optionally when relevant, the present value and the past values extracted from the electronic medical record of the patient related to the blood creatinine level, the arterial pressure, the heart rate and the electrocardiogram, the body temperature, the oxygen saturation, the respiratory rate, the weight of the patient, amounts of fluids administered to the patient and the current diseases.

10. The prediction method of diuresis according to claim 9, wherein the adaptive mathematical model is a model, wherein a calibration algorithm of the adaptive mathematical model considers available additional information relevant to the patient provided in real time, through a use of Bayesian estimators.

11. The prediction method of diuresis according to claim 10, wherein the predicted values referred to at step 303 are relevant to corresponding time instants increased, wherein each increment is a temporal value ranging from 5 minutes to 6 hours.

12. The prediction method of diuresis according to claim 11, wherein the machine learning mathematical model is selected from regression models with a variable dichotomous response comprising logit and probit models and machine learning models comprising classification models, artificial neural networks and SVM models.

13. A monitoring system of a biological fluid for predicting a state of health of a patient, comprising:
a biological fluid container;
a weight meter of the biological fluid container;
a device comprising a first algorithm for recording, storing, comparing and processing measurements of the biological fluid container and a second algorithm for predicting future measurements of the biological fluid container and the state of health of the patient associated with the future measurements of the biological fluid container;
a videoterminal for displaying outputs of the first algorithm and outputs of the second algorithm present in the device;
a first “wireless” system for connecting the weight meter and the device; and
a second “wireless” system for connecting the device and the videoterminal.

14. The monitoring system according to claim 13, wherein the biological fluid is selected from a peritoneal fluid, a lymphatic fluid, a urine, a blood, an amniotic fluid and a saliva.

15. The monitoring system according to claim 14, wherein the biological fluid container is a sterile bag.

16. A monitoring method of a biological fluid for predicting a state of health of a patient, comprising the following steps:
step 200) taking a sample of the biological fluid produced by the patient in a predetermined period of time and collecting the biological fluid in the biological fluid container;
step 201 weighing the biological fluid container;
step 202) by a first algorithm, recording and storing measurements of the biological fluid container;
step 203) repeating the previous steps, from step 200 to step 202, for a predetermined number of times;
step 204) by the first algorithm, comparing and processing the measurements of the biological fluid container recorded and stored over time to determine a trend of an organic fluid weight;
on a basis of the trend determined in step 204, by a second algorithm comprising an adaptive mathematical model and a machine learning mathematical model, predicting values of future measurements of the biological fluid container and a risk of worsening of health conditions of the patient; and
step 206) transferring data obtained in step 205, to a videoterminal.

17. The monitoring method according to claim 16, wherein the biological fluid is selected from a peritoneal fluid, a lymphatic fluid, a urine, a blood, an amniotic fluid and a saliva.

18. The monitoring method according to claim 17, wherein:
the first algorithm comprises a mathematical model for an analysis of the data obtained through a weight meter to correlate each weight measurement with the time instant and to calculate a rate hourly production of biological fluid normalized on a weight of the patient, wherein the analysis was performed in the time instant;
the second algorithm comprises:
an adaptive mathematical model having as an input at least a present value and past values of a biological fluid flow as calculated by the first algorithm and, when relevant, the present value and the past values extracted from an electronic medical record of the patient and having as an output predictions of future container weight measurements;
a first mathematical model for comparing the predictions of the future container weight measurements with corresponding values observed in real time;
a second mathematical model for correcting a calculation performed by the adaptive mathematical model on a basis of a comparison result; and
a third mathematical model having as an input an output of the adaptive mathematical model, a present value and past values of weight measurements of the biological fluid container and physiological parameters present in the electronic medical record of the patient, and having as an output a risk level ranging from 1 to 10 of worsening of health of the patient in 24/48 hours after a last weight measurement of the biological fluid container.

19. The monitoring method according to claim 18, wherein:
the predetermined period of time referred to at step 200 ranges from 30 seconds to 10 minutes, or the predetermined period of time is equal to 5 minutes; and
the predetermined number of times referred to at step 203 ranges from 1 to 100, or the predetermined number of times is equal to 50.

20. A prediction method of a biological fluid flow for calculating a level of a state of health of a patient, comprising the following steps:
step 400) by an adaptive mathematical model, calculating a trend of a biological fluid of the patient considering
at least a present value and past values of the biological fluid as recorded and processed by a device and
optionally when relevant, the present value and the past values extracted from an electronic medical record of the patient related to a blood creatinine level, an arterial pressure, a heart rate and an electrocardiogram, a body temperature, an oxygen saturation, a respiratory rate, a weight of the patient, amounts of fluids administered to the patient and current diseases;
step 401) comparing an expected value, wherein a calculation output referred to step 400, with corresponding values observed in real time;
step 402) correcting a calculation referred to at step 400 on a basis of a comparison referred to in step 401;
step 403) by a machine learning mathematical model, calculating the level of the state of health in future instants considering:
at least the present value, the past values and values predicted by the adaptive mathematical model of the biological fluid and
optionally when relevant, the present value and the past values extracted from the electronic medical record of the patient related to the blood creatinine level, the arterial pressure, the heart rate and the electrocardiogram, the body temperature, the oxygen saturation, the respiratory rate, the weight of the patient, amounts of fluids administered to the patient and the current diseases.

21. The prediction method according to claim 20, wherein the adaptive mathematical model is a model, wherein a calibration algorithm considers available additional information relevant to the patient provided in real time, preferably through a use of Bayesian estimators.

22. The prediction method according to claim 21, wherein predicted values referred to at step 403 are related to corresponding time instants, incremented so that each increment is a temporal value ranging from 5 minutes to 6 hours.

23. The prediction method of biological fluid flow according to claim 22, wherein the machine learning mathematical model is selected from regression models with variable dichotomous response comprising logit and probit models and machine learning models comprising classification models, artificial neural networks and SVM models.

24. The prediction method of biological fluid flow according to claim 23, wherein the biological fluid is selected from a peritoneal fluid, a lymphatic fluid, a urine, a blood, an amniotic fluid and a saliva.
